(19) United States
(12) Patent Application Publication
(10) Pub. No.: US 2024/0308945 A1
(43) Pub. Date: Sep. 19, 2024

(54) Synthetic Cannabinoid Compounds, Pharmaceutical Compositions, and Methods of Treating Anxiety and Other Disorders

(71) Applicant: Miralogx LLC, Tampa, FL (US)

(72) Inventor: Jonnie R. Williams, SR., Sarasota, FL (US)

(21) Appl. No.: 18/672,845

(22) Filed: May 23, 2024

Related U.S. Application Data
(63) Continuation-in-part of application No. PCT/US2022/050757, filed on Nov. 22, 2022.
(60) Provisional application No. 63/626,870, filed on Jan. 30, 2024.
(60) Provisional application No. 63/403,544, filed on Sep. 2, 2022.
(60) Provisional application No. 63/283,431, filed on Nov. 27, 2021.

(51) Int. Cl. C07C 39/19; A61K 31/00; C07D 311/94

(52) U.S. Cl. CPC C07C 39/19; A61K 31/658; C07D 311/94

(57) ABSTRACT

Cannabinoid analogs may exhibit anti-inflammatory properties such as by inhibition of cannabinoid type 2 (CB₂) receptors. Pharmaceutical compositions comprising the cannabinoid analogs may be used to treat various diseases and conditions in mammals, including pain, anxiety, addiction, epilepsy, depression, or Alzheimer's disease.

### CROSS-REFERENCE TO RELATED APPLICATIONS

This application is a continuation-in-part of International Application No. PCT/US2022/050757, filed Nov. 22, 2022, which claims priority under 35 U.S.C. § 119(e) to U.S. App. No. 63/403,544, filed Sep. 2, 2022, and U.S. App. No. 63/283,431, filed Nov. 27, 2021. This application also claims priority under 35 U.S.C. § 119(e) to U.S. App. No. 63/626,870, filed Jan. 30, 2024. Each of these applications is hereby incorporated by reference in its entirety.

### BACKGROUND

There has been considerable research in recent years on the therapeutic effects of cannabis including its constituents tetrahydrocannabinol (THC) and cannabidiol (CBD). There remains a need for improved compounds for treating disorders such as depression, anxiety, substance addiction, pain, cancer, autoimmune disorders, and other disorders associated with chronic inflammation. It would be particularly desirable to develop compounds which may be prepared synthetically and formulated as solid oral dosage forms.

### SUMMARY

According to one aspect, a synthetic cannabinoid analog has a structure of Formula (I):

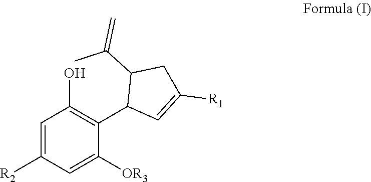

wherein R₁ is selected from the group consisting of H, OH, protected hydroxyl, alkyl, alkenyl, alkynyl, acyl, aryl, heteroaryl, cycloalkyl, and heterocycle; wherein the alkyl, alkenyl, alkynyl or acyl is optionally substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl;
R₂ is C₆₋₁₀ alkyl;
R₃ is selected from the group consisting of H, alkyl, acyl, —SO₂-alkyl, —SO₂-aryl and —SO₂-heteroaryl; wherein the alkyl is optionally substituted with one or more substituents independently selected from the group consisting of halogen, —OH, alkyl, —O-alkyl, NR^{E}R^{F}, —S-alkyl, —SO-alkyl, —SO₂-alkyl, aryl and heteroaryl; and wherein R^{E} and R^{F} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, NR^{G}R^{H}, —S-alkyl, —SO-alkyl and —SO₂-alkyl; wherein R^{G} and R^{H} are each independently selected from hydrogen and C₁₋₄ alkyl;
   or a pharmaceutically acceptable salt or ester thereof.

According to another aspect, a method of treating a cancer, tumor, addiction, epilepsy, anxiety, or depression comprises administering to an individual in need thereof a pharmaceutical composition comprising a therapeutically effective amount of a synthetic cannabinoid analog of Formula (I) and a pharmaceutically acceptable carrier therefor.

In another aspect, a synthetic cannabinoid analog has a structure of Formula (II):

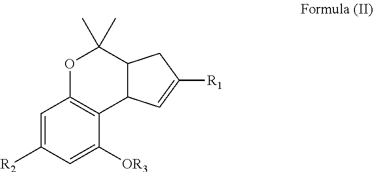

wherein R₁, R₂, and R₃ are as previously defined, or a pharmaceutically acceptable salt or ester thereof.

In another aspect, a synthetic cannabinoid analog has the structure:

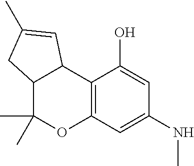

or a pharmaceutically acceptable salt or ester thereof.

In another aspect, method of treating anxiety, addiction, depression, or Alzheimer's disease comprises administering to an individual in need thereof a pharmaceutical composition comprising a therapeutically effective amount of a synthetic cannabinoid analog of Formula (II) and a pharmaceutically acceptable carrier therefor.

In another aspect, a pharmaceutical composition comprises a therapeutically effective amount of a synthetic cannabinoid analog of Formula (I) or (II), or a combination thereof, and a pharmaceutically acceptable carrier therefor.

In some embodiments, the compounds disclosed herein can be used in a method of treating cognitive decline, for example in elderly patients.

### BRIEF DESCRIPTION OF THE DRAWINGS

FIG. 1 is the HPLC chromatogram for Batch A of the synthesized compound 7-heptyl-2,4,4-trimethyl-3,3a,4,9b-tetrahydrocyclopenta[c]chromen-9-ol (“M251”).

FIG. 2 is the mass spectrum for Batch A of 7-heptyl-2,4,4-trimethyl-3,3a,4,9b-tetrahydrocyclopenta[c]chromen-9-ol (“M251”).

FIG. 3 is the ¹H-NMR and ¹³C-NMR spectrums for Batch A of 7-heptyl-2,4,4-trimethyl-3,3a,4,9b-tetrahydrocyclopenta[c]chromen-9-ol (“M251”).

FIG. 4 shows thermogravimetric (TGA) analysis of Batch A of 7-heptyl-2,4,4-trimethyl-3,3a,4,9b-tetrahydrocyclopenta[c]chromen-9-ol (“M251”).

FIG. 5 is the HPLC chromatogram for Batch B of the synthesized compound 7-heptyl-2,4,4-trimethyl-3,3a,4,9b-tetrahydrocyclopenta[c]chromen-9-ol (“M251”).

FIG. 6 is the mass spectrum for Batch B of 7-heptyl-2,4,4-trimethyl-3,3a,4,9b-tetrahydrocyclopenta[c]chromen-9-ol (“M251”).

FIG. 7 is the ¹H-NMR and ¹³C-NMR spectrums for Batch B of 7-heptyl-2,4,4-trimethyl-3,3a,4,9b-tetrahydrocyclopenta[c]chromen-9-ol (“M251”).

FIG. 8 shows thermogravimetric (TGA) analysis of Batch B of 7-heptyl-2,4,4-trimethyl-3,3a,4,9b-tetrahydrocyclopenta[c]chromen-9-ol (“M251”).

### DETAILED DESCRIPTION

Cannabinoids produced by the Cannabis sativa plant have the potential to treat a vast assortment of diseases and other human ailments. More than 100 different cannabinoids have been isolated from cannabis and each cannabinoid compound exhibits various effects. For example, THC is well-known for its psychological effects and CBD is known for its non-psychoactive effects. THC and related derivatives typically exert therapeutic activities via cannabinoid receptors found in humans and other mammals. CBD is an isomer of THC. CBD and CBD derivatives also exhibit anti-oxidative and anti-inflammatory effects through pathways not related to cannabinoid receptors. Cannabinoid type 1 (CB₁) receptors are found primarily in the brain, including the basal ganglia and in the limbic system, and the hippocampus and the striatum, as well as the cerebellum. CB₁ receptors can be found in the human anterior eye and retina. Research indicates that cannabinoid type 2 (CB₂) receptors are responsible for anti-inflammatory and other therapeutic effects related to cannabinoids.

Cannabis plants that contain high levels of cannabinoids such as THC, for example, are typically known as “marijuana” plants. Cannabis plants with a low cannabinoid content are categorized as “hemp” plants. Individual countries usually determine the levels of cannabinoids that differentiate between cannabis plants that are categorized as marijuana or hemp plants. Generally, the THC content on a dry-weight basis for a cannabis plant categorized as a hemp plant is 0.3% or less. Cannabis sativa plants having THC, CBD, and other cannabinoid content levels greater than 0.3% are typically considered marijuana plants. Medical marijuana typically contains cannabinoid levels between 5 and 20%. Other Cannabis sativa plants may produce cannabinoid levels from 25 to 30%.

The biosynthetic pathway of the Cannabis sativa plant that produces the various cannabinoids starts with the precursor cannabigerolic acid. The enzymes THCA synthase and CBDA synthase catalyze the biosynthesis of cannabigerolic acid to tetrahydrocannabinol acid (THCA) and cannabidiol acid (CBDA), respectively, as well as other cannabinoids. It is known that various other cannabinoids are produced via this pathway. THC, CBD, and other cannabinoid derivatives are generated artificially from THCA and CBDA by non-enzymatic decarboxylation. Aizpurua-Olaizola et al., “Evolution of the Cannabinoid and Terpene Content during the Growth of Cannabis sativa Plants from Different Chemotypes,” J. Natural Prods. 2016 79 (2), 324-331. Various classes of cannabinoids are biosynthesized via this general pathway to include THC (tetrahydrocannabinol), THCA (tetrahydrocannabinolic acid), CBD (cannabidiol), CBDA (cannabidiolic Acid), CBN (cannabinol), CBG (cannabigerol), CBC (cannabichromene), CBL (cannabicyclol), CBV (cannabivarin), (tetrahydrocannabivarin), CBDV (cannabidivarin), CBCV (cannabichromevarin), CBGV (cannabigerovarin), CBGM (cannabigerol monomethyl ether), CBE (cannabielsoin), and CBT (cannabicitran).

### 1. Synthetic Cannabinoid Analogs

According to some aspects, a synthetic cannabinoid analog has a structure of Formula (I):

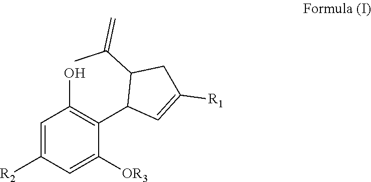

wherein R₁ is selected from the group consisting of H, OH, protected hydroxyl, alkyl, alkenyl, alkynyl, acyl, aryl, heteroaryl, cycloalkyl, and heterocycle; wherein the alkyl, alkenyl, alkynyl or acyl is optionally substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl;
R₂ is C₆₋₁₀ alkyl;
R₃ is selected from the group consisting of H, alkyl, acyl, —SO₂-alkyl, —SO₂-aryl and —SO₂-heteroaryl; wherein the alkyl is optionally substituted with one or more substituents independently selected from the group consisting of halogen, —OH, alkyl, —O-alkyl, NR^{E}R^{F}, —S-alkyl, —SO-alkyl, —SO₂-alkyl, aryl and heteroaryl; and wherein R^{E} and R^{F} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, NR^{G}R^{H}, —S-alkyl, —SO-alkyl and —SO₂-alkyl; wherein R^{G} and R^{H} are each independently selected from hydrogen and C₁₋₄ alkyl;
   or a pharmaceutically acceptable salt or ester thereof.

In another aspect, a synthetic cannabinoid analog has a structure of Formula (II):

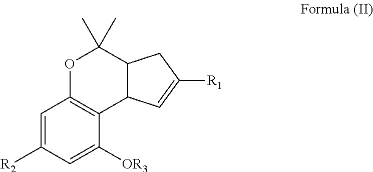

wherein R₁, R₂, and R₃ are as previously defined, or a pharmaceutically acceptable salt or ester thereof.

In another aspect, a synthetic cannabinoid analog has the structure:

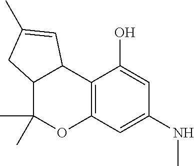

or a pharmaceutically acceptable salt or ester thereof.

As used herein the term “alkyl,” whether alone or as part of a substituent group, refers to a saturated C₁-Cₙ carbon chain, wherein the carbon chain may be straight or branched; wherein n can be 2, 3, 4, 5, 6, 7, 8, 9 or 10. Suitable examples include, but are not limited to methyl, ethyl, propyl, isopropyl, butyl, isobutyl, sec-butyl, t-butyl, n-pentyl and n-hexyl.

As used herein the term “alkenyl,” whether alone or as part of a substituent group, refers to a C₂-Cₙ carbon chain, wherein the carbon chain may be straight or branched, wherein the carbon chain contains at least one carbon-carbon double bond, and wherein n can be 3, 4, 5, 6, 7, 8, 9 or 10.

As used herein the term “alkynyl,” whether alone or as part of a substituent group, refers to a C₂-Cₙ, wherein the carbon chain may be straight or branched, wherein the carbon chain contains at least one carbon-carbon triple bond, and wherein n can be 3, 4, 5, 6, 7, 8, 9 or 10.

As used herein the term “aryl,” whether alone or as part of a substituent group, refers to an unsubstituted carboxylic aromatic ring comprising between 6 to 14 carbon atoms. Suitable examples include, but are not limited to, phenyl and naphthyl.

As used herein the term “protected hydroxyl” refers to a hydroxyl group substituted with a suitably selected oxygen protecting group. More particularly, a “protected hydroxyl” refers to a substituent group of the formula —OPG₁ wherein PG₁ is a suitably selected oxygen protecting group. During any of the processes for preparation of the compounds of the present disclosure it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein the term “oxygen protecting group” refers to a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM and THP. Other suitable oxygen protecting groups may be found in texts such as T. W. Greene & P. G. M. Wuts, Protective Groups in Organic Synthesis, John Wiley & Sons, 1991.

As used herein the term “nitrogen protecting group” refers to a group which may be attached to a nitrogen atom to protect said nitrogen atom from participating in a reaction and which may be readily removed following the reaction. Suitable nitrogen protecting groups include, but are not limited to, carbamates groups of the formula —C(O)—OR wherein R can be methyl, ethyl, t-butyl, benzyl, phenylethyl, CH₂═CH—CH₂—, and the like; amide groups of the formula —C(O)—R′ wherein R′ can be methyl, phenyl, trifluoromethyl, and the like; N-sulfonyl derivative groups of the formula —SO₂—R″ wherein R″ can be tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein the term “acyl” refers to a group of the formula —CO—Cₙ wherein Cₙ represent a straight or branched alkyl chain wherein n can be 1, 2, 3, 4, 5, 6, 7, 8, 9 or 10.

As used herein the term “heteroaryl” refers to any five or six membered monocyclic aromatic ring structure containing at least one heteroatom selected from the group consisting of O, N and S, and optionally containing one to three additional heteroatoms independently selected from the group consisting of O, N and S; or a nine or ten membered bicyclic aromatic ring structure containing at least one heteroatom selected from the group consisting of O, N and S, and optionally containing one to four additional heteroatoms independently selected from the group consisting of O, N and S. The heteroaryl group may be attached at any heteroatom or carbon atom of the ring such that the result is a stable structure. Examples of suitable heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl and pteridinyl.

As used herein the term “cycloalkyl” refers to any monocyclic ring containing from four to six carbon atoms, or a bicyclic ring containing from eight to ten carbon atoms. The cycloalkyl group may be attached at any carbon atom of the ring such that the result is a stable structure. Examples of suitable cycloalkyl groups include, but are not limited to, cyclopropyl, cyclobutyl, cyclopentyl and cyclohexyl.

As used herein the term “heterocycle” refers to any four to six membered monocyclic ring structure containing at least one heteroatom selected from the group consisting of O, N and S, and optionally containing one to three additional heteroatoms independently selected from the group consisting of O, N and S; or an eight to ten membered bicyclic ring structure containing at least one heteroatom selected from the group consisting of O, N and S, and optionally containing one to four additional heteroatoms independently selected from the group consisting of O, N and S. The heterocycle group may be attached at any heteroatom or carbon atom of the ring such that the result is a stable structure. Examples of suitable heterocycle groups include, but are not limited to, azetidine, azete, oxetane, oxete, thietane, thiete, diazetidine, diazete, dioxetane, dioxete, dithietane, dithiete, pyrrolidine, pyrrole, tetrahydrofuran, furan, thiolane, thiophene, piperidine, oxane, thiane, pyridine, pyran and thiopyran.

The groups described herein can be unsubstituted or substituted, as herein defined. In addition, the substituted groups can be substituted with one or more groups such as a C₁-C₆ alkyl, C₁₋₄ alkyl, —O—C₁₋₄ alkyl, hydroxyl, amino, (C₁₋₄ alkyl)amino, di(C₁₋₄ alkyl)amino, —S—(C₁₋₄ alkyl), —SO—(C₁₋₄ alkyl), —SO₂—(C₁₋₄ alkyl), halogen, aryl, heteroaryl, and the like.

With reference to substituents, the term “independently” means that when more than one of such substituents is possible, such substituents may be the same or different from each other.

The compounds of the present disclosure may contain at least one hydroxyl group. These at least one hydroxyl group may form an ester with inorganic or organic acid. In particular, pharmaceutically acceptable acids. The ester(s) may form chiral carbons. The present disclosure is directed toward all stereo-chemical forms of the compounds of the present disclosure, including those formed by the formation of one or more ester groups.

### II. Non-Limiting Examples of Synthetic Cannabinoid Analogs in Accordance With the Present Disclosure

Non-limiting examples of synthetic cannabinoid analogs according to Formula I are illustrated below:

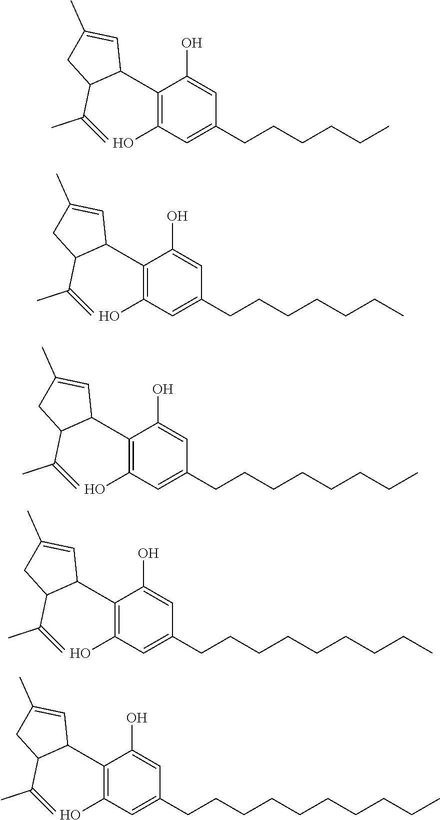

Non-limiting examples of synthetic cannabinoid analogs according to Formula II are illustrated below:

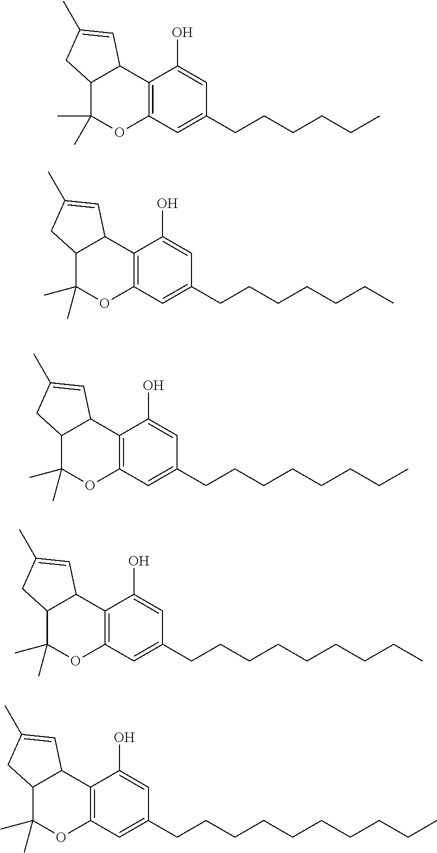

### III. Synthesis and Purification of the Cannabinoid Compounds

The compounds described herein may be prepared synthetically using known techniques with appropriate modifications to the reactants to form the structures shown herein or by other suitable pathways that will be apparent to persons skilled in the art. By way of non-limiting example, compounds described herein may be synthesized according to one or more of the following pathways described in Razdan, Total Synthesis of Cannabinoids, SISA Incorporated, Cambridge, Massachusetts, with appropriate modifications to the reactants, as will be apparent to persons skilled in the art, to yield the structures disclosed herein. Alternatively, the synthesis techniques described in Dialer et al. U.S. Pat. No. 10,059,683 B2, the disclosure of which is hereby incorporated by reference in its entirety, may be suitably adapted to synthesize the cannabinoid analogs described herein.

In some examples, the cannabinoid compounds described herein may be formed as salts, which may be helpful to improve chemical purity, stability, solubility, and/or bioavailability. Non-limiting examples of possible salts are described in P. H. Stahl et al., Handbook of Pharmaceutical Salts: Properties, Selection and Use, Weinheim/Zürich: Wiley-VCH/VHCA, 2002, including salts of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid.

Compounds intended for administration to humans or other mammals generally should have very high purity. In the case of synthetically prepared compounds, purity refers to the ratio of a compound's mass to the total sample mass following any purification steps. Usually, the level of purity is at least about 95%, more usually at least about 96%, about 97%, about 98%, or higher. For example, the level of purity may be about 98.5%, 99.0%, 99.1%, 99.2%, 99.3%, 99.4%, 99.5%, 99.6%, 99.7%, 99.8%, 99.9%, or higher.

Compounds described herein that exist in more than one optical isomer form (enantiomers) may be provided either as racemic mixture or by isolating one of the enantiomers, the latter case in which purity as described above may refer to enantiomeric purity.

### IV. Methods of Use

As described above, cannabinoids and related cannabinoid analogs typically exert therapeutic and anti-inflammatory activities via CB₂ cannabinoid receptors. While not wanting to be bound by theory, compounds disclosed herein may also exhibit properties as inhibitors of monoamine oxidase (MAO) activity, including either or both of MAO-A and MAO-B activity. These properties may enable compounds to be effective for treating indications associated with MAO activity, such as depression, pain, substance addiction, smoking cessation, and the like. Hence, in some embodiments, the compounds disclosed herein can be used in a method of treating diseases and conditions associated with monoamine oxidase (MAO) activity. In some embodiments, the individual suffers from depression, pain, or addiction.

Compounds disclosed herein also (or alternatively) may exhibit anti-inflammatory properties owing to the compound's interaction with inflammation pathways, including by way of example, interleukins such as IL-1 and IL-6, TNF-α, cyclooxygenase (COX), and the like. A compound's ability to inhibit MAO-A and/or MAO-B activity, and/or its ability to inhibit COX and/or other pathways associated with inflammation may be evaluated using assays well known to persons of ordinary skill in the art.

In some aspects, a cannabinoid analog as described herein is administered to an individual in need thereof for the treatment of a substance addiction, such as alcohol, tobacco, opioid, prescription drugs, cocaine, benzodiazepines, amphetamines, hallucinogens, inhalants, phencyclidine, or other drug addictions. Such treatments also are inclusive of treating withdrawal in dependency on benzodiazepines, opiates, or alcohol, as well as symptoms experienced by patients with substance use disorders, such as anxiety, mood symptoms, pain, and insomnia.

In addition to anxiety that is associated with substance use disorders, the cannabinoid analogs may be effective for treating other types of anxiety disorders, such as post-traumatic stress disorder, general anxiety disorder, panic disorder, social anxiety disorder, and obsessive-compulsive disorder.

In other aspects, a cannabinoid analog as described herein may be administered to an individual in need thereof for the treatment of multiple sclerosis, fibromyalgia, epilepsy or neuropsychiatric disorders that are linked to epilepsy, such as neurodegeneration, neuronal injury, and psychiatric diseases. The cannabinoid analogs may be effective for potentiating the anticonvulsant activity of other active agents such as phenytoin and diazepam.

In still other aspects, a cannabinoid analog as described herein may be used in as an antipsychotic for treating patients with schizophrenia. The cannabinoid analogs also may be effective to reduce intraocular pressure, such as in the treatment of glaucoma.

In yet other aspects, a cannabinoid analog as described herein may be administered to an individual in need thereof for the treatment of cancer. The cannabinoid analog may be effective to block cancer cells from spreading around the body and invading an area entirely; for suppressing the growth of cancer cells and/or promoting the death of cancer cells.

The cannabinoid analogs as described herein may be useful in the treatment of Type 1 diabetes, which is caused by inflammation when the immune system attacks cells in the pancreas; as well as acne, which is caused, in part, by inflammation and overworked sebaceous glands on the body. The anti-inflammatory properties of the compounds may lower the production of sebum that leads to acne, including acne vulgaris, the most common form of acne.

The cannabinoid analogs as described herein may be used to treat Alzheimer's disease, and particularly to prevent the development of social recognition deficit in subjects when administered in the early stages of Alzheimer's disease. Other examples of disorders that may be treated by the cannabinoid analog as described herein include nausea, vomiting, anorexia, and cachexia. The compounds may produce an appetite-enhancing effect, for example in AIDS patients or individuals with Alzheimer's disease who refuse food.

The cannabinoid analogs as described herein may be useful in the treatment of spasticity caused by multiple sclerosis (MS) or spinal cord injury, movement disorders, such as Tourette's syndrome, dystonia, or tardive dyskinesia. MS patients may experience benefits on ataxia and reduction of tremors.

Analgesic properties of the cannabinoid analogs may prove beneficial, for example, in the treatment of neuropathic pain due to multiple sclerosis, damage of the brachial plexus and HIV infection, pain in rheumatoid arthritis, cancer pain, headache, menstrual pain, chronic bowel inflammation and neuralgias.

The cannabinoid analogs as described herein may be useful in the treatment of asthma. Experiments examining the anti-asthmatic effect of THC or cannabis date mainly from the 1970s, and are all acute studies. The effects of a cannabis cigarette (2% THC) or oral THC (15 mg), respectively, approximately correspond to those obtained with therapeutic doses of common bronchodilator drugs (salbutamol, isoprenaline). Since inhalation of cannabis products may irritate the mucous membranes, oral administration or another alternative delivery system would be preferable. Very few patients developed bronchoconstriction after inhalation of THC.

An improvement of mood in reactive depression has been observed in several clinical studies with THC. There are additional case reports claiming benefit of cannabinoids in other psychiatric symptoms and diseases, such as sleep disorders, anxiety disorders, bipolar disorders, and dysthymia. Various authors have expressed different viewpoints concerning psychiatric syndromes and cannabis. While some emphasize the problems caused by cannabis, others promote the therapeutic possibilities. Quite possibly cannabis products may be either beneficial or harmful, depending on the particular case. The attending physician and the patient should be open to a critical examination of the topic, and a frankness to both possibilities.

In a number of painful syndromes secondary to inflammatory processes (e.g. ulcerative colitis, arthritis), cannabis products may act not only as analgesics but also demonstrate anti-inflammatory potential. For example, some patients employing cannabis report a decrease in their need for steroidal and nonsteroidal anti-inflammatory drugs. Moreover there are some reports of positive effects of cannabis self-medication in allergic conditions. It is as yet unclear whether cannabis products may have relevant effects on causative processes of autoimmune diseases.

There are a number of positive patient reports on medical conditions that cannot be easily assigned to the above categories, such as pruritus, hiccup, ADS (attention deficit syndrome), high blood pressure, tinnitus, chronic fatigue syndrome, restless leg syndrome, and others. Different authors have described several hundred possible indications for cannabis and THC. For example, 2.5 to 5 mg THC were effective in three patients with pruritus due to liver diseases. Another example is the successful treatment of a chronic hiccup that developed after a surgery. No medication was effective, but smoking of a cannabis cigarette completely abolished the symptoms.

Cannabis products often show very good effects in diseases with multiple symptoms that encompassed within the spectrum of THC effects, for example, in painful conditions that have an inflammatory origin (e.g., arthritis), or are accompanied by increased muscle tone (e.g., menstrual cramps, spinal cord injury), or in diseases with nausea and anorexia accompanied by pain, anxiety and depression, respectively (e.g. AIDS, cancer, hepatitis C).

COVID-19 is transmitted through respiratory droplets and uses receptor-mediated entry into a human host via angiotensin-converting enzyme II (ACE2) that is expressed in lung tissue, as well as oral and nasal mucosa, kidney, testes, and the gastrointestinal tract. Modulation of ACE2 levels in these gateway tissues may decrease disease susceptibility. See Wang et al., In Search of Preventative Strategies: Novel Anti-Inflammatory High-CBD Cannabis sativa Extracts Modulate ACE2 Expression in COVID-19 Gateway Tissues (Apr. 17, 2020), doi: 10.20944/preprints202004.0315.v1. The cannabinoid analogs as described herein may modulate ACE2 expression and may have utility in the treatment of a coronavirus such as COVID-19.

### V. Dosage and Pharmaceutical Compositions

Suitable doses may vary over a wide range depending on a variety of factors including the type and/or severity of the disease or disorder, previous treatments, the general health, age, and/or weight of the individual, the frequency of treatments, the rate of release from the composition, and other diseases present. This dose may vary according to factors such as the disease state, age, and weight of the subject. For example, higher doses may be administered for treatments involving conditions that are at an advanced stage and/or life threatening. Dosage regimens also may be adjusted to provide the optimum therapeutic response.

Pharmaceutical compositions may be formulated together with one or more acceptable pharmaceutical or food grade carriers or excipients. As used herein, the term “acceptable pharmaceutical or food grade carrier or excipient” means a non-toxic, inert solid, semisolid or liquid filler, diluent, encapsulating material or formulation auxiliary of any type. For example, sugars such as lactose, glucose and sucrose; starches such as corn starch and potato starch; cellulose and its analogs such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions may be prepared by any suitable technique and is not limited by any particular method for its production. For example, purified cannabinoids can be combined with excipients and a binder, and then granulated. The granulation can be dry-blended with any remaining ingredients, and compressed into a solid form such as a tablet.

Pharmaceutical compositions may be administered by any suitable route. For example, the compositions may be administered orally, parenterally, by inhalation spray, topically, rectally, nasally, buccally, vaginally, via an implanted reservoir, or ingested as a dietary supplement or food. In some embodiments, a composition is provided in an inhaler, which may be actuated to administer a vaporized medium that is inhaled into the lungs. The term parenteral as used herein includes subcutaneous, intracutaneous, intravenous, intramuscular, and intracranial injection or infusion techniques. Most often, the pharmaceutical compositions are readily administered orally and ingested.

Pharmaceutical compositions may contain any conventional non-toxic pharmaceutically-acceptable carriers, adjuvants or vehicles. In some cases, the pH of the formulation may be adjusted with acceptable pharmaceutical or food grade acids, bases or buffers to enhance the stability of the formulated composition or its delivery form.

Liquid dosage forms for oral administration include acceptable pharmaceutical or food grade emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylsulfoxide (DMSO) dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, lozenges, pills, powders, and granules. In such solid dosage forms, the active compound is mixed with at least one inert, acceptable pharmaceutical or food grade excipient or carrier such as sodium citrate or dicalcium phosphate and/or a) fillers or extenders such as starches, lactose, sucrose, glucose, mannitol, and silicic acid, b) binders such as carboxymethylcellulose, alginates, gelatin, polyvinylpyrrolidone, sucrose and acacia, c) humectants such as glycerol, d) disintegrating agents such as agaragar, calcium carbonate, potato or tapioca starch, alginic acid, certain silicates, and sodium carbonate, e) solution retarding agents such as paraffin, f) absorption accelerators such as quaternary ammonium compounds, g) wetting agents such as cetyl alcohol and glycerol monostearate, h) absorbents such as kaolin and bentonite clay, i) lubricants such as talc, calcium stearate, magnesium stearate, solid polyethylene glycols, sodium lauryl sulfate, and mixtures thereof, and j) sweetening, flavoring, perfuming agents, and mixtures thereof. In the case of capsules, lozenges, tablets and pills, the dosage form may also comprise buffering agents.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract or, optionally, in a delayed or extended manner. Examples of embedding compositions which can be used include polymeric substances and waxes. Tablet formulations for extended release are also described in U.S. Pat. No. 5,942,244.

Compositions may contain a cannabinoid analog or compounds, alone or with other therapeutic compound(s). A therapeutic compound is a compound that provides pharmacological activity or other direct effect in the diagnosis, cure, mitigation, treatment, or prevention of disease, or to affect the structure or any function of the body of man or animals. A therapeutic compound disclosed herein may be used in the form of a pharmaceutically acceptable salt, solvate, or solvate of a salt, e.g., a hydrochloride. Additionally, therapeutic compound disclosed herein may be provided as racemates, or as individual enantiomers, including the R- or S-enantiomer. Thus, the therapeutic compound disclosed herein may comprise a R-enantiomer only, a S-enantiomer only, or a combination of both a R-enantiomer and a S-enantiomer of a therapeutic compound. In some aspects, the therapeutic compound may have anti-inflammatory activity, such as a non-steroidal anti-inflammatory drug (NSAID). NSAIDs are a large group of therapeutic compounds with analgesic, anti-inflammatory, and anti-pyretic properties. NSAIDs reduce inflammation by blocking cyclooxygenase. NSAIDs include, without limitation, aceclofenac, acemetacin, actarit, alcofenac, alminoprofen, amfenac, aloxipirin, aminophenazone, antraphenine, aspirin, azapropazone, benorilate, benoxaprofen, benzydamine, butibufen, celecoxib, chlorthenoxacin, choline salicylate, clometacin, dexketoprofen, diclofenac, diflunisal, emorfazone, epirizole; etodolac, etoricoxib, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidin, loxoprofen, lumiracoxib, mefenamic acid, meloxicam, metamizole, metiazinic acid, mofebutazone, mofezolac, nabumetone, naproxen, nifenazone, niflumic acid, oxametacin, phenacetin, pipebuzone, pranoprofen, propyphenazone, proquazone, protizinic acid, rofecoxib, salicylamide, salsalate, sulindac, suprofen, tiaramide, tinoridine, tolfenamic acid, valdecoxib, and zomepirac.

NSAIDs may be classified based on their chemical structure or mechanism of action. Non-limiting examples of NSAIDs include a salicylate derivative NSAID, a p-amino phenol derivative NSAID, a propionic acid derivative NSAID, an acetic acid derivative NSAID, an enolic acid derivative NSAID, a fenamic acid derivative NSAID, a non-selective cyclooxygenase (COX) inhibitor, a selective cyclooxygenase-1 (COX-1) inhibitor, and a selective cyclooxygenase-2 (COX-2) inhibitor. An NSAID may be a profen. Examples of a suitable salicylate derivative NSAID include, without limitation, acetylsalicylic acid (aspirin), diflunisal, and salsalate. Examples of a suitable p-amino phenol derivative NSAID include, without limitation, paracetamol and phenacetin. Examples of a suitable propionic acid derivative NSAID include, without limitation, alminoprofen, benoxaprofen, dexketoprofen, fenoprofen, flurbiprofen, ibuprofen, indoprofen, ketoprofen, loxoprofen, naproxen, oxaprozin, pranoprofen, and suprofen. Examples of a suitable acetic acid derivative NSAID include, without limitation, aceclofenac, acemetacin, actarit, alcofenac, amfenac, clometacin, diclofenac, etodolac, felbinac, fenclofenac, indometacin, ketorolac, metiazinic acid, mofezolac, nabumetone, naproxen, oxametacin, sulindac, and zomepirac. Examples of a suitable enolic acid (oxicam) derivative NSAID include, without limitation, droxicam, isoxicam, lornoxicam, meloxicam, piroxicam, and tenoxicam. Examples of a suitable fenamic acid derivative NSAID include, without limitation, flufenamic acid, mefenamic acid, meclofenamic acid, and tolfenamic acid. Examples of a suitable selective COX-2 inhibitors include, without limitation, celecoxib, etoricoxib, firocoxib, lumiracoxib, meloxicam, parecoxib, rofecoxib, and valdecoxib.

A therapeutically effective amount of a therapeutic compound disclosed herein generally is in the range of about 0.001 mg/kg/day to about 100 mg/kg/day. An effective amount may be, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day. In some examples, an effective amount of a therapeutic compound may be in the range of about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day. In other examples, an effective amount of a therapeutic compound disclosed herein may be in the range of, e.g., about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day.

In addition to pharmaceutical compositions, compounds described herein may be formulated as an elixir, a beverage, a chew, a tablet, a lozenge, a gum, or the like. According to another aspect, the pharmaceutical compositions may also be formulated as a pharmaceutically acceptable vehicle such as a capsule, tablet, syrup, lozenge, inhaler, e-cigarette, chewable gum, nasal spray, transdermal patch, liquid, transmucosal vehicle, hydrogel, nanosome, liposome, noisome, nanoparticle, nanosphere, microsphere, microparticle, microemulsion, nanosuspension, or micelle. The compositions may also be formulated, for example, as dietary supplements or nutraceuticals.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLE 1

This example illustrates the synthesis of 7-heptyl-2,4,4-trimethyl-3,3a,4,9b-tetrahydrocyclopenta[c]chromen-9-ol (“M251”), whose structure is shown below.

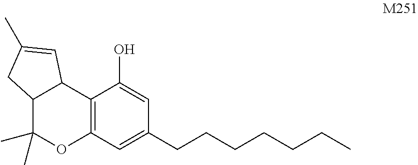 [SMILES: CCCCCCCc1cc(O)c2c(c1)OC(C)(C)C1CC(C)=CC21]

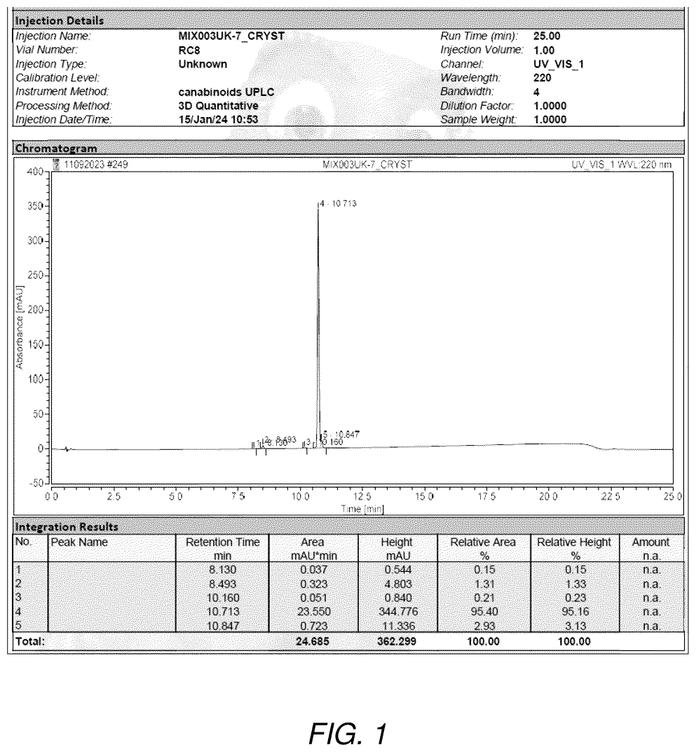

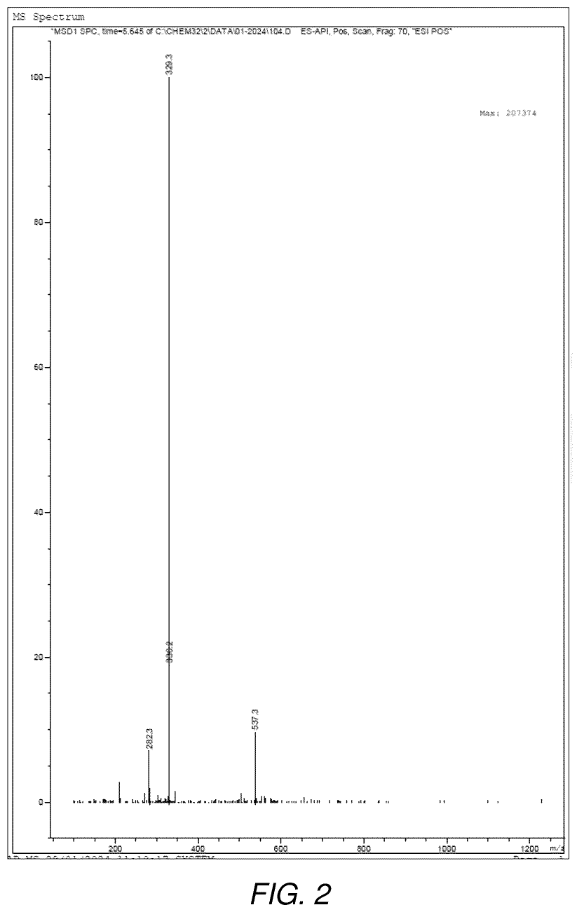

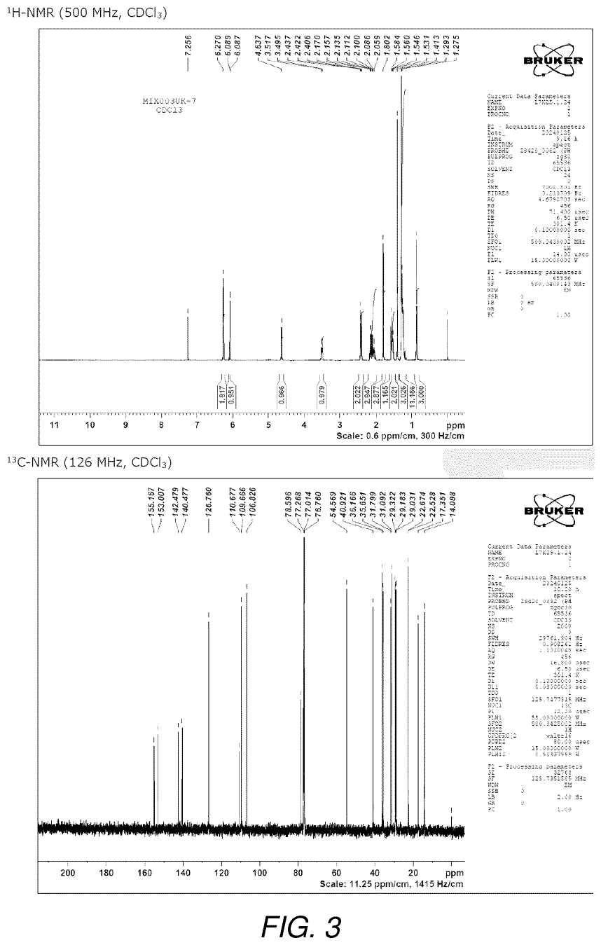

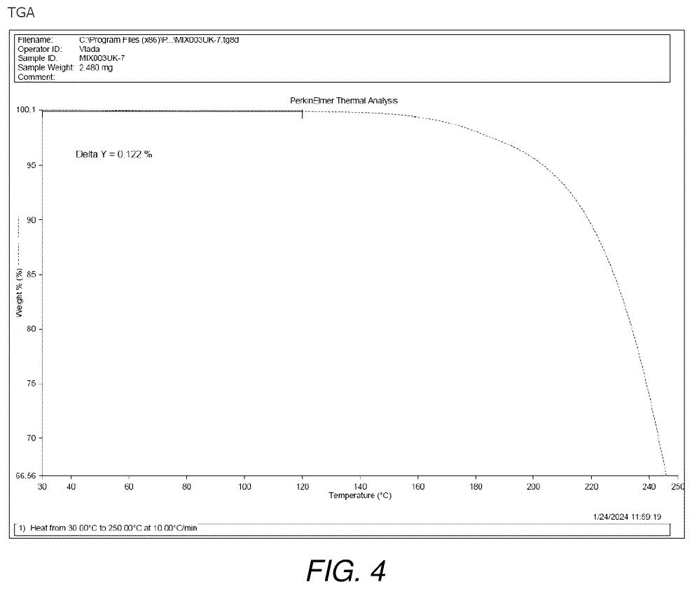

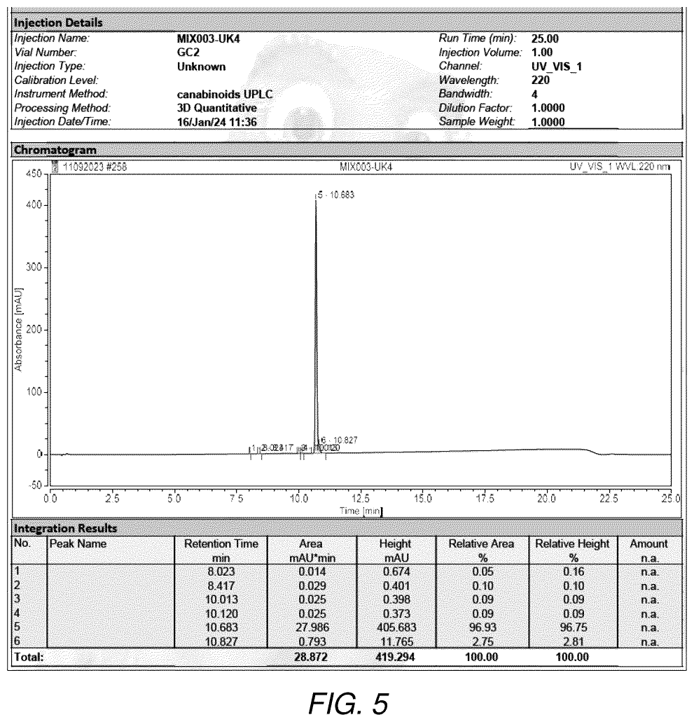

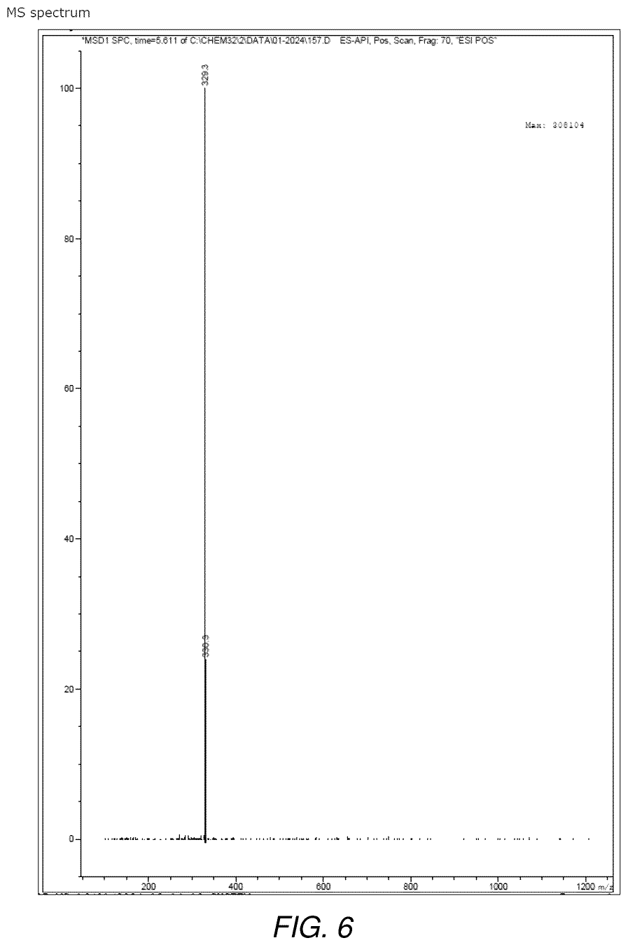

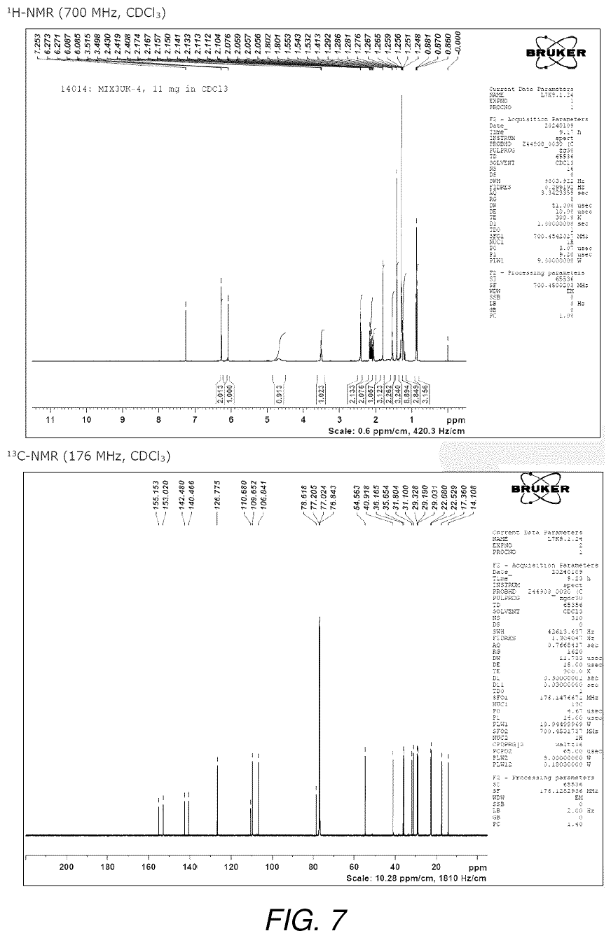

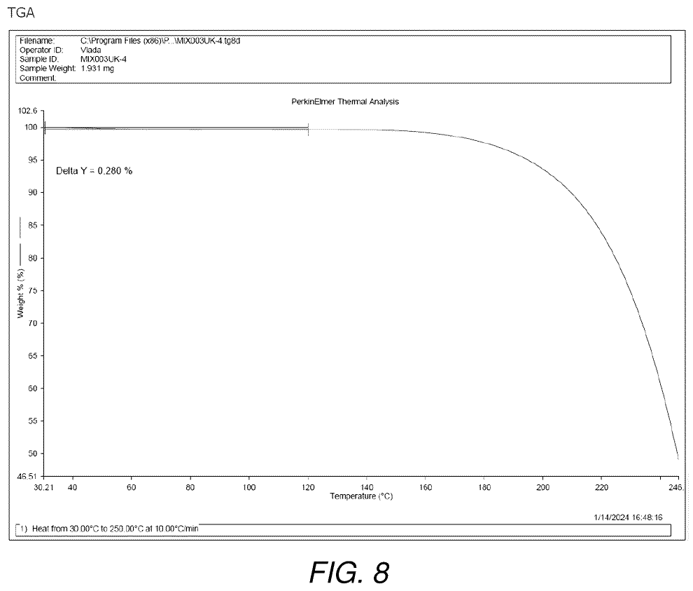

Step 1. A mixture of cyclopent-2-enone (200 g, 2.44 mol, 1 eq) and NBS (490 g, 2.76 mol, 1.13 eq) in CHCl₃ (3.9 L) was heated in 6L reactor to 62° C. (jacket temp 64° C.) and stirred (130 rpm) under reflux for several hours. When the starting material was fully consumed, the mixture was cooled and filtered. The filtrate was washed with water (1 L), dried over MgSO₄ (80 g), filtered, and used in Step 2 without evaporation.
Step 2. The solution from Step 1 (theoretical 392 g, 2.43 mol, 1 eq) in CHCl₃ (3.9 L) was heated to 30° C. Sn reagent (1209 g, 3.65 mol, 1.5 eq) was charged into a pressure-equalizing funnel, 1/3 of the amount (400 g, 0.5 eq) was added to the mixture, followed by Pd(OAc)₂ (27 g, 0.12 mol, 0.05 eq) dissolved in CHCl₃ (300 mL). When the isothermal reaction slowed down, the rest of Sn reagent and Pd(OAc)₂ (27 g, 0.12 mol, 0.05 eq) dissolved in CHCl₃ (300 mL) were added to the mixture (the rate of addition was 20-30 minutes, the end of the addition was simultaneous) and the stirring was continued for 2 hours. Then, the reaction was quenched by addition of 2.5% K₂CO₃ (2 L), and the phases were separated. The organic phase was placed in 2L-flask and concentrated, then water (1 L) was added and the obtained mixture was partially evaporated (0.5 L) to the receiving flask and removed to Erlenmeyer. An additional amount of water was added and the steam evaporation was continued (1 L) until no starting material remained in the crude mixture. The water +product phases were combined (total 1.5 L) and extracted with MTBE (300 mL), and the organic phase was evaporated and distilled to provide a pure product (67.7 g, ˜30% yield for Steps 1+2).
Step 3—Batch A. The isolated material from Step 2 (3 g, 24.5 mmol, 1 eq) was dissolved in THF (extra dry, 60 mL) and the obtained solution was cooled to −40° C. A solution of MeMgCl (3M in THF, 12.5 mL, 36 mmol, 1.45 eq) was added dropwise at −40° C. to −35° C. The stirring was continued for about 1 hour until the starting material was fully consumed. The reaction was quenched by slow addition of sat. NH₄Cl (60 mL) at −40° C. and the mixture was heated to room temperature. An organic phase was separated, and the water layer was washed with pentane (50 mL). The organic phase was washed with brine, dried over Na₂SO₄, filtrated, and evaporated at 13° C. to up to 6 g (theoretical 100% yield ˜3.4 g).
Step 3—Batch B. The isolated material from Step 2 (64.7 g, 0.554 mol, 1 eq) was dissolved in THF (extra dry, 1.35 L) and the obtained solution was cooled to −37° C. A solution of MeMgCl (3M in THF, 280 mL, 0.84 mol, 1.5 eq) was added dropwise in 4 portions (after each portion the mixture was cooled to −37° C.). The stirring was continued for about 4 hours until the starting material was fully consumed. The reaction was quenched by slow addition of sat. NH₄Cl (125 g in 1 L) at −35° C. and the mixture was heated to room temperature. An organic phase was separated, and the water layer was washed with pentane (600 mL). The organic phase was washed with brine (2×300 mL), dried over Na₂SO₄ (150 g), filtrated, and evaporated at 13° C. (bath) at 330-45 mbar to give 90 g (100% theoretical yield).
Step 4—Batch A. Heptyl-resercinol (10.2 g, 0.05 mol, 2 eq) was dissolved in DCM (100 mL), then dry PTSA (240 mg, 1.39 mmol, 5% eq) and MgSO₄ (10 g) were introduced. The suspension was cooled to 4° C. and the product from Step 3, Batch A (theoretical 3.4 g, 0.025 mol, 1 eq) in DCM (30 mL) was added dropwise for 10 minutes. The reaction mixture was stirred for an additional 30 minutes after the stop of addition and quenched by the addition of sat. NaHCO₃ solution. The phases were separated and the organic solution was dried over Na₂SO₄, filtered, and concentrated under reduced pressure to provide 15.3 g of the crude product as a brown oil. The crude product was purified by Silica gel chromatography and eluted with 5-20% DCM/hexane. The pure product (97% HPLC purity) was isolated as a yellowish oil (1.9 g, ˜24% yield for 2 steps).
Step 4—Batch B. Heptyl-resercinol (13.4 g, 0.064 mol, 1.5 eq) was dissolved in DCM (150 mL), then dry PTSA (315 mg, 1.78 mmol, 5% eq) and MgSO₄ (15 g) were introduced. The suspension was cooled to 4° C. and the product of Step 3, Batch B (theoretical 6.6 g, 0.042 mol, 1 eq) in DCM (80 mL) was added dropwise for 20 minutes. The mixture was stirred for an additional 30 minutes after the stop of addition and quenched by the addition of sat. NaHCO₃ solution. The phases were separated and the organic solution was dried over Na₂SO₄, filtered, and concentrated under reduced pressure to provide 21.3 g of the crude product as a brown oil. The crude product was purified by Silica gel chromatography and eluted with 5-20% DCM/hexane. The pure product (96% HPLC purity) was isolated as a yellowish oil (1.75 g after purification).
Step 5—Batch A. The product of Step 4, Batch A (1.7 g, 5.2 mmol, 1 eq) was dissolved in toluene (55 mL, 33 mL/g) and dry PTSA (50 mg, 0.28 mmol, 5% eq) was introduced. The obtained mixture was stirred at 35° C. and monitored. After 2 hours conversion of 75% was observed and 10% NaHCO₃ (50 mL) was added, the phases were separated. The organic layer was washed with water, 1% citric acid, and brine, dried over Na₂SO₄, and evaporated at 22° C. to afford 1.7 g of the crude product as a yellow-green oil. The crude product was purified by Silica gel column chromatography (eluted with 5-20% DCM/hexane) and recrystallized from pentane to afford 214 mg of M251 as a white solid with HPLC purity of 95.40%. FIG. 1 shows the results of analysis by HPLC. FIG. 2 shows the results of analysis by mass spectroscopy. FIG. 3 shows the results of analysis by ¹H-NMR and ¹³C-NMR. FIG. 4 shows the results of thermogravimetric analysis (TGA).
Step 5—Batch B. The product of Step 4, Batch B (1.9 g, 5.8 mmol, 1 eq) was dissolved in toluene (63 mL, 33 mL/g) and dry PTSA (55 mg, 0.31 mmol, 5% eq) was introduced. The obtained reaction mixture was stirred at 35° C. and monitored. After 4 hours, a conversion of 75% was observed and 10% NaHCO₃ (50 mL) was added, the phases were separated. The organic layer was washed with water, 1% citric acid, and brine, dried over Na₂SO₄, and evaporated at 22° C. to afford 1.9 g of the crude product as a yellow-green oil. The crude product was purified by Silica gel column chromatography (eluted with 5-20% DCM/hexane) and recrystallized from pentane to afford 140 mg of M251 as a white solid with HPLC purity of 96.93% after purification and recrystallization. FIG. 5 shows the results of analysis by HPLC. FIG. 6 shows the results of analysis by mass spectroscopy. FIG. 7 shows the results of analysis by ¹H-NMR and ¹³C-NMR. FIG. 8 shows the results of thermogravimetric analysis (TGA).

### EXAMPLE 2

This example illustrates evaluating 7-heptyl-2,4,4-trimethyl-3,3a,4,9b-tetrahydrocyclo-penta[c]chromen-9-ol (M251, Example 1) in silico for activity with respect to a number of targets including cannabinoid type 1 (CB₁) and cannabinoid type 2 (CB₂) receptors. The study was performed by InSilicoTrials Technologies SpA.

The activity prediction algorithm takes a query ligand and for each target in the database provides an activity probability distribution in terms of two parameters: the most probable value and the variance. To this effect it compares the query ligand with each of the ligands in the database for which the activity is experimentally well characterized in the database. The importance of each such ligand and its experimental activity values is related to the similarity of the ligand with the query molecule and characterized by the precision value τ=1/σ2, where σ is the standard deviation.

All the τ values for each ligand concur to the determination of a global τ value. The availability of experimental values determines which kind of activity measure is predictable and with which precision. The database considers quantitative prediction Ki, Kd, IC₅₀ and EC₅₀ values. For simplicity, only the best activity precision, annotated with the associated activity type is reported.

The targets for which the prediction is sufficiently accurate are reported alongside the available predictions and the _84 . While activities are reported in molarity units, the σ value refers to the decimal logarithm of the activity. This means that, within one sigma, the activity range in logarithm scale is [log 10 A−σ, log 10 A+σ], which in natural scale, corresponds to [A·10−σ, A·10σ]. For example, σ=1 and activity A=3 nM implies that within one sigma the activity is in the range [0.3 nM, 30.0 nM]. Table 1 below presents the outcome of the bioactivity-based screening, ranked according to the values accounting for the precision of the prediction.

TABLE 1

| Target | IC₅₀ | | EC₅₀ | | Ki | | Kd | | σ_{P} |
|---|---|---|---|---|---|---|---|---|---|
| Cannabinoid CB1 receptor: Rattus | 32.9 | nM | 21.8 | nM | 36.5 | nM | 19.0 | nM | 1.2: Ki |
| norvegicus | | | | | | | | | |
| Cannabinoid CB2 receptor: Mus musculus | 612 | nM | 27.6 | nM | 23.7 | nM | | | 1.2: Ki |
| Cannabinoid CB1 receptor: Homo sapiens | 80.5 | nM | 163 | nM | 102 | nM | 8.97 | nM | 1.2: Ki |
| Cannabinoid CB2 receptor: Homo sapiens | 290 | nM | 44.1 | nM | 60.0 | nM | 6.86 | nM | 1.3: Ki |
| Vascular endothelial growth factor receptor | 151 | nM | 65.3 | nM | 101 | nM | 120 | nM | 1.4: Ki |
| 2: Homo sapiens | | | | | | | | | |
| Dopamine D3 receptor: Homo sapiens | 113 | nM | 10.7 | nM | 31.3 | nM | 10.4 | nM | 1.5: EC₅₀ |
| Transient receptor potential cation channel | 1.71 | μM | 1.90 | μM | | | | | 1.5: EC₅₀ |
| subfamily A member 1: Rattus norvegicus | | | | | | | | | |
| Cannabinoid CB1 receptor: Mus musculus | 96.2 | nM | 267 | nM | 72.9 | nM | 55.7 | nM | 1.6: Ki |
| L-lactate dehydrogenase A chain: Homo | 2.04 | μM | 849 | nM | 5.02 | μM | 6.00 | μM | 1.6: Ki |
| sapiens | | | | | | | | | |
| G-protein coupled receptor 55: Homo | 3.53 | μM | 1.19 | μM | | | | | 1.6: EC₅₀ |
| sapiens | | | | | | | | | |
| N-arachidonyl glycine receptor: Homo | 4.69 | μM | 2.14 | μM | | | | | 1.7: EC₅₀ |
| sapiens | | | | | | | | | |
| Pregnane X receptor: Homo sapiens | 1.04 | μM | 2.65 | μM | 201 | nM | | | 1.7: EC₅₀ |
| Glycine receptor subunit alpha-1: Homo | 755 | nM | 599 | nM | 103 | nM | | | 1.8: EC₅₀ |
| sapiens | | | | | | | | | |
| Anandamide amidohydrolase: Homo sapiens | 136 | nM | 14.2 | nM | 28.5 | nM | 0.80 | nM | 1.8: IC₅₀ |
| Monoacylglycerol lipase ABHD6: Homo | 344 | nM | | | 3.41 | μM | | | 1.8: IC₅₀ |
| sapiens | | | | | | | | | |
| Monoacylglycerol lipase ABHD12: Homo | 5.80 | μM | | | | | | | 1.8: IC₅₀ |
| sapiens | | | | | | | | | |
| Sn1-specific diacylglycerol lipase alpha: | 209 | nM | | | 700 | nM | | | 2: IC₅₀ |
| Homo sapiens | | | | | | | | | |
| Transient receptor potential cation channel | 2.27 | μM | 4.62 | μM | | | | | 2: EC₅₀ |
| subfamily V member 2: Rattus norvegicus | | | | | | | | | |
| Vanilloid receptor: Homo sapiens | 135 | nM | 475 | nM | 5.68 | nM | 44.9 | nM | 2: EC₅₀ |
| Transient receptor potential cation channel | 1.23 | μM | 1.84 | μM | | | | | 2: IC₅₀ |
| subfamily M member 8: Rattus norvegicus | | | | | | | | | |
| Transient receptor potential cation channel | 168 | nM | 4.89 | μM | | | | | 2: IC₅₀ |
| subfamily V member 4: Rattus norvegicus | | | | | | | | | |
| Cannabinoid CB2 receptor: Rattus | 133 | nM | 16.9 | nM | 17.1 | nM | | | 2.1: Ki |
| norvegicus | | | | | | | | | |
| Kappa opioid receptor: Homo sapiens | 530 | nM | 36.8 | nM | 37.0 | nM | 1.66 | nM | 2.1: Ki |
| Mu opioid receptor: Homo sapiens | 228 | nM | 103 | nM | 40.2 | nM | 0.73 | nM | 2.1: Ki |
| Delta opioid receptor: Homo sapiens | 65.1 | nM | 42.5 | nM | 102 | nM | 24.1 | nM | 2.1: Ki |
| Cytochrome P450 1B1: Homo sapiens | 161 | nM | 6.05 | μM | 642 | nM | | | 2.2: Ki |
| Cytochrome P450 1A1: Homo sapiens | 426 | nM | 9.58 | μM | 900 | nM | 5.26 | μM | 2.2: Ki |
| Cytochrome P450 1A2: Homo sapiens | 3.13 | μM | 49.7 | μM | 701 | nM | | | 2.2: Ki |
| Transient receptor potential cation channel, | 10.1 | μM | 5.33 | μM | | | | | 2.2: EC₅₀ |
| subfamily V, member 3: Rattus norvegicus | | | | | | | | | |
| Dopamine D1 receptor: Homo sapiens | 698 | nM | 53.5 | nM | 130 | nM | 849 | nM | 2.3: Ki |

IC₅₀: Inhibitory concentration at 50% of the maximum effect; EC₅₀: Concentration at 50% of the maximum response; Ki: Equilibrium inhibition constant; Kd: Equilibrium dissociation constant. The value of σ is a measure of the deviation of the prediction, with respect to the most relevant prediction (IC₅₀, EC₅₀, Ki or Kd) and provided as decimal logarithm of the value. Example: σ = 1 and activity A = 3 nM implies that within one sigma the activity is in the range [0.3 nM, 30.0 nM].

While the invention has been described with respect to specific examples. those skilled in the art will appreciate that there are numerous variations and permutations of the above described systems and techniques that fall within the spirit and scope of the invention as set forth in the appended claims.

What is claimed is:

1. A compound having a structure of Formula (I): 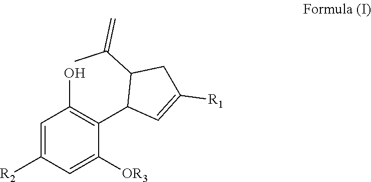
wherein R₁ is selected from the group consisting of H, OH, protected hydroxyl, alkyl, alkenyl, alkynyl, acyl, aryl, heteroaryl, cycloalkyl, and heterocycle; wherein the alkyl, alkenyl, alkynyl or acyl is optionally substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl;
R₂ is C₆₋₁₀ alkyl;
R₃ is selected from the group consisting of H, alkyl, acyl, —SO₂-alkyl, —SO₂-aryl and —SO₂-heteroaryl; wherein the alkyl is optionally substituted with one or more substituents independently selected from the group consisting of halogen, —OH, alkyl, —O-alkyl, NR^{E}R^{F}, —S-alkyl, —SO-alkyl, —SO₂-alkyl, aryl and heteroaryl; and wherein R^{E} and R^{F} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, NR^{G}R^{H}, —S-alkyl, —SO-alkyl and —SO₂-alkyl; wherein R^{G} and R^{H} are each independently selected from hydrogen and C₁₋₄ alkyl;
or a pharmaceutically acceptable salt or ester thereof.

2. A pharmaceutical composition comprising the compound of claim 1 and a pharmaceutically acceptable vehicle therefor.

3. A pharmaceutical composition of claim 2, wherein the pharmaceutically acceptable vehicle is selected from the group consisting of a capsule, tablet, syrup, lozenge, inhaler, chewable gum, nasal spray, transdermal patch, liquid, transmucosal vehicle, hydrogel, nanosome, liposome, noisome, nanoparticle, nanosphere, microsphere, microparticle, microemulsion, nanosuspension, and micelle.

4. A method of treating a cancer, tumor, addiction, epilepsy, anxiety, pain or depression comprising administering to an individual in need thereof the pharmaceutical composition of claim 3.

5. A compound of claim 1 which has a structure selected from the group consisting of: 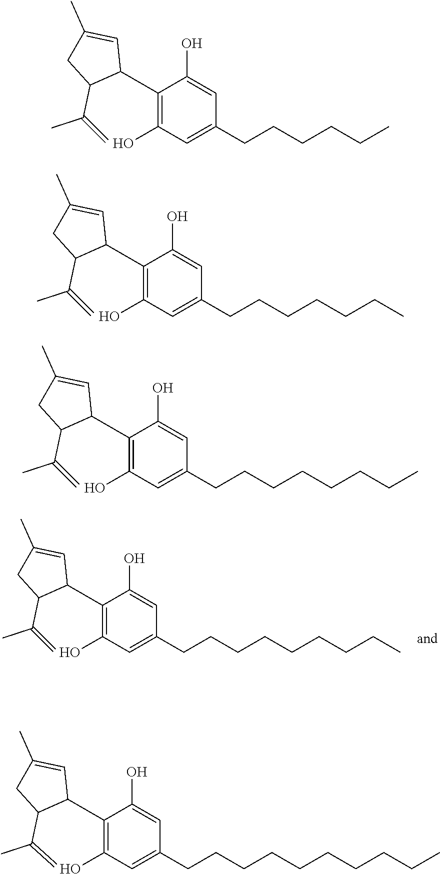
or a pharmaceutically acceptable salt thereof.

6. A pharmaceutical composition comprising the compound of claim 5 and a pharmaceutically acceptable vehicle therefor.

7. A method of treating a cancer, tumor, addiction, epilepsy, anxiety, pain or depression comprising administering to an individual in need thereof the pharmaceutical composition of claim 6.

8. A compound having a structure of Formula (II): 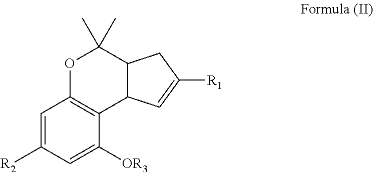
wherein R₁ is selected from the group consisting of H, OH, protected hydroxyl, alkyl, alkenyl, alkynyl, acyl, aryl, heteroaryl, cycloalkyl, and heterocycle; wherein the alkyl, alkenyl, alkynyl or acyl is optionally substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl;
R₂ is C₆₋₁₀ alkyl;
R₃ is selected from the group consisting of H, alkyl, acyl, —SO₂-alkyl, —SO₂-aryl and —SO₂-heteroaryl; wherein the alkyl is optionally substituted with one or more substituents independently selected from the group consisting of halogen, —OH, alkyl, —O-alkyl, NR^{E}R^{F}, —S-alkyl, —SO-alkyl, —SO₂-alkyl, aryl and heteroaryl; and wherein R^{E} and R^{F} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, NR^{G}R^{H}, —S-alkyl, —SO-alkyl and —SO₂-alkyl; wherein R^{G} and R^{H} are each independently selected from hydrogen and C₁₋₄ alkyl;
or a pharmaceutically acceptable salt or ester thereof.

9. A pharmaceutical composition comprising the compound of claim 8 and a pharmaceutically acceptable vehicle therefor.

10. A pharmaceutical composition of claim 9, wherein the pharmaceutically acceptable vehicle is selected from the group consisting of a capsule, tablet, syrup, lozenge, inhaler, chewable gum, nasal spray, transdermal patch, liquid, transmucosal vehicle, hydrogel, nanosome, liposome, noisome, nanoparticle, nanosphere, microsphere, microparticle, microemulsion, nanosuspension, and micelle.

11. A method of treating anxiety, addiction, pain or depression comprising administering to an individual in need thereof the pharmaceutical composition of claim 10.

12. A method of treating Alzheimer's disease comprising administering to an individual in need thereof the pharmaceutical composition of claim 10.

13. The compound of claim 8 which has a structure selected from the group consisting of: 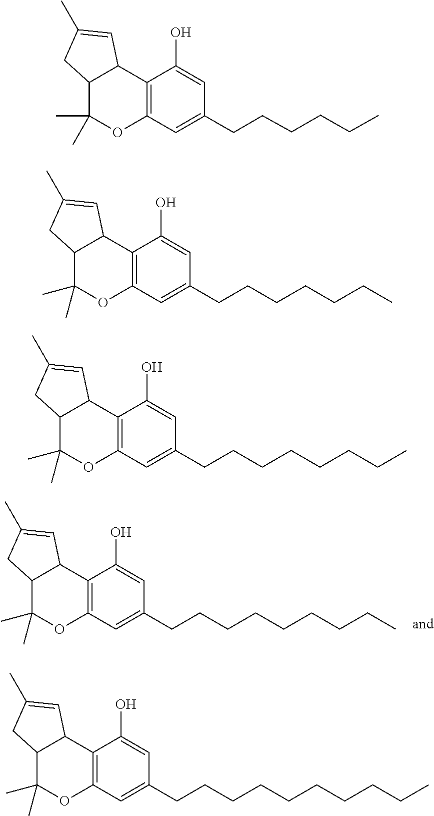
or a pharmaceutically acceptable salt thereof.

14. A pharmaceutical composition comprising the compound of claim 13 and a pharmaceutically acceptable vehicle therefor.

15. A method of treating a cancer, tumor, addiction, epilepsy, anxiety, pain or depression comprising administering to an individual in need thereof the pharmaceutical composition of claim 14.

16. The compound of claim 8 which is 7-heptyl-2,4,4-trimethyl-3,3a,4,9b-tetrahydrocyclopenta[c]chromen-9-ol.

17. A pharmaceutical composition comprising the compound of claim 16 and a pharmaceutically acceptable vehicle therefor.

18. A pharmaceutical composition of claim 17, wherein the pharmaceutically acceptable vehicle is selected from the group consisting of a capsule, tablet, syrup, lozenge, inhaler, chewable gum, nasal spray, transdermal patch, liquid, transmucosal vehicle, hydrogel, nanosome, liposome, noisome, nanoparticle, nanosphere, microsphere, microparticle, microemulsion, nanosuspension, and micelle.

19. A method of treating anxiety, addiction, pain or depression comprising administering to an individual in need thereof the pharmaceutical composition of claim 18.

20. A method of treating Alzheimer's disease comprising administering to an individual in need thereof the pharmaceutical composition of claim 18.
